(19) United States
(12) Patent Application Publication
(10) Pub. No.: US 2025/0255755 A1
(43) Pub. Date: Aug. 14, 2025

(54) WOUND HEALING DEVICE AND METHODS FOR WOUND HEALING

(71) Applicant: SINUS CLEAR INNOVATIONS, LLC, BILOXI, MS (US)

(72) Inventors: David Burkart, Biloxi, MS (US); Clayton Bratton, Biloxi, MS (US); Vincent Pisciotta, Biloxi, MS (US)

(21) Appl. No.: 18/896,473

(22) Filed: Sep. 25, 2024

Related U.S. Application Data
(63) Continuation of application No. 16/649,235, filed on Mar. 20, 2020, now abandoned.
(60) Provisional application No. 62/561,057, filed on Sep. 20, 2017.

(51) Int. Cl. A61F 13/36; A61F 13/12; A61F 13/38; A61K 31/7048; A61K 31/7056; A61K 38/12

(52) U.S. Cl. CPC A61F 13/36; A61F 13/126; A61F 13/385; A61K 31/7048; A61K 31/7056; A61K 38/12

(57) ABSTRACT

The present invention is directed to a therapeutic device to promote wound healing and a kit for promotion of wound healing. A method of use of the therapeutic device is also disclosed herein.

This application claims priority from U.S. Provisional Patent Application No. 62/561,057, filed on Sep. 20, 2017, the contents of which are incorporated herein by reference in its entirety.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

Not applicable.

### FIELD OF THE INVENTION

The present invention is directed to a device, a kit, and associated methods for targeted delivery of therapeutic compounds. In some embodiments, the present invention promotes wound healing and prevents infection.

### BACKGROUND OF THE INVENTION

Wound healing is a complex process of tissue repair in response to bodily injury. A disruption in the normal healing process can lead to scar formation or the development of chronic wounds that can require up to several years to fully close. According the Wound Healing Society, chronic wounds affect 6.5 million patients in the United States alone, amounting to an annual economic burden of an estimated $25 billion. Furthermore, the incidence of chronic wounds increases with age. Thus, as the population ages over the coming years, the economic burden of chronic wounds is only expected to increase.

The process of wound healing can be generally broken down into four phases. The first phase of wound healing is known as hemostasis, which generally comprises the body's response to stop the bleeding associated with the injury. The second phase is inflammation, wherein antibodies, white blood cells, and macrophages clean the wound by killing bacteria and clearing debris. The inflammation phase is often associated with burning, itching, and pain. Proliferation represents the third phase of wound healing and is characterized by rebuilding of healthy tissue within the wound. The final phase, maturation, occurs after the wound has closed and consists of fortification of the new tissue.

Wound packing can be performed to speed the healing process and reduce the likelihood of infection. The process generally comprises filling the wound with a moistened wound dressings and replacing the wound dressings after a certain amount of time has passed. This process is repeated until the wound is clean and begins to close.

In some instances, therapeutic compounds are introduced during the wound-healing process, which can increase the frequency of wound dressing changes. Importantly, the majority of wound packing occurs during the inflammation and proliferation stages, when the wound is most tender. Thus, the extensive and repetitive process of traditional wound packing can be very painful and can result in improper packing if the associated pain is too great.

Therefore, a simplified therapeutic device to promote wound healing that avoids the tedious process of traditional wound packing while simultaneously delivering therapeutic compounds is needed.

### SUMMARY OF THE INVENTION

In one aspect, the present invention provides a therapeutic device to promote wound healing. In this aspect, the therapeutic device comprises wound-packing material configured to hold one or more therapeutic inserts; a non-adherent, absorbent coating that surrounds at least a portion of the wound-packing material; a plurality of therapeutic inserts configured to reside within the wound-packing material; or a combination thereof.

The therapeutic device can be configured to reside within or adjacent to a wound or surgical site. In embodiments, the therapeutic device is configured to reside within a bodily cavity. By way of non-limiting examples, embodiments can be configured to reside within the nose, the mouth, the ear, or any other bodily cavity. In one embodiment, the therapeutic device is configured to reside within the nasal cavity. In alternate embodiments, the therapeutic device is configured to be applied to any of various surface wounds or lesions known in the art. Examples of such surface wounds include, but are not limited to avulsions, decubiti, warts, cancerous or pre-cancerous growths, tumors, blemishes, or any other surface wound known to those of skill in the art. The device can be configured to cover relatively large surface wounds that extend through the epidermis, dermis, subcutaneous tissue, muscle, or a combination thereof. In embodiments, the device is configured to facilitate healing of wounds located on the torso, neck, head, face, extremities, or a combination thereof. The device can promote healing of wounds in the peroneal area. In embodiments, the therapeutic device is configured to permit debridement of the wound site during scheduled dressing changes.

The wound-packing material can comprise various substances that are appropriate for absorbing drainage from a wound. In one embodiment, the wound-packing material comprises a polyvinyl alcohol (PVA) sponge. In an alternate embodiment, the wound-packing material comprises a collagen dressing to stimulate tissue growth during the proliferation stage of wound healing.

In embodiments, the non-adherent, absorbent coating is designed to completely surround and enclose the wound-packing material. In alternate embodiments, the non-adherent coating is applied to a single surface of the therapeutic device. The non-adherent, absorbent coating is configured to prevent the therapeutic device from adhering to a wound during the healing process. Embodiments can also comprise a low-adherence coating. Examples of non-adherent or low-adherence coatings include, but are not limited to, Tefla™ Dressings available from Medtronics Minimally Invasive Therapies (Minneapolis, MN), Medi-Pack Performance Non-Adherent Dressing available from Mckesson (San Francisco, CA), and Adapatic Dressing available from Systagenix (Gatwick, United Kingdom).

Embodiments of the therapeutic device are provided in various shapes and sizes. The therapeutic device can be generally cylindrical in design with substantially parallel sides. In cylindrical embodiments, the cross-section of the therapeutic device can be substantially circular, substantially oval, or polygonal in shape. In polygonal embodiments, the cross section can comprise shapes that are regular or irregular polygons. Embodiments of the therapeutic device are substantially triangular in shape, substantially rectangular in shape, or substantially square. In certain embodiments, the therapeutic device is substantially disc-shaped. Embodiments can comprise straight or curved edges.

In one aspect, the shape of the therapeutic device is customizable. By way of non-limiting examples, the shape can be varied depending on the wound's size, location, or type. The therapeutic device can also be shaped according to the subject or patient's needs or preferences, including, but not limited, to the subject's age, the subject's comfort, or the subject's lifestyle. In certain embodiments, the therapeutic device is customizable to fit within or on a large surface wound or lesion. There can be a number of alternate or additional considerations in customizing the appropriate size and shape of the therapeutic device that can be apparent to one having skill in the art.

In embodiments, the wound-packing material comprises a plurality of perforations configured to receive one or more therapeutic inserts. The perforations can be in any form that is appropriate for receiving and holding the therapeutic inserts. The perforations can comprise gaps, holes, slots, notches, grooves, or a combination thereof. The perforations can be designed to extend partially or completely across a longitudinal or horizontal length of the wound-packing material.

The therapeutic inserts can be reversibly or irreversibly coupled to the wound-packing material. In embodiments, the therapeutic inserts are obtained or provided separately from the therapeutic device. The therapeutic inserts can be attached to the wound-packing material via any mechanism known to one of skill in the art. By way of non-limiting example, the therapeutic inserts can be sutured to the wound-packing material, bound to the wound-packing material with a bio adhesive agent, or frictionally secured within or on the wound-packing material.

The therapeutic insert can comprise of various materials and can exist in a variety of sizes and shapes. In embodiments, the therapeutic insert is structurally rigid. The therapeutic insert can comprise a disc, rod, cone, or any other shape. In embodiments, the therapeutic insert can be pliable to allow for customization of the design of the therapeutic device. In embodiments, the therapeutic inserts are comprised of a biodegradable or bio-absorbable polymer material. Examples of such a material include, but are not limited to polyethylene glycol, poly trimethylene carbonate, polycaprolactone, self-reinforced polyglycolide, or self-reinforced polylactide. In certain embodiments, the therapeutic inserts are comprised of glyceride, synthetic glyceride, semi-synthetic glyceride, or a combination thereof.

In an alternate aspect, the wound-packing material comprises one or more pockets, openings, or cavities configured to receive a therapeutic mixture, wherein the mixture further comprises one or more therapeutic compounds. In embodiments, the pockets, openings, or cavities include a tapering that terminates in a narrowed portion at one end of the pocket, opening, or cavity. The narrowed portion serves as a port, a channel, or a point of injection to allow for introduction of the therapeutic mixture into the pocket, opening, or cavity of the therapeutic device. In certain embodiments, the therapeutic mixture is substantially liquid before introduction into the therapeutic device. The liquid therapeutic mixture can also include a solidifying agent that is configured to promote solidification of the therapeutic mixture after the mixture is introduced into the pockets, openings, or cavities of the therapeutic device. In embodiments, the solidified therapeutic mixture forms a suppository-type substance within the therapeutic device that is configured to melt or dissolve upon contact with a wound to allow controlled release of therapeutic compounds into the wound. The solidifying agent can be comprised of any agent, compound, or material known by skilled artisan or later developed that promotes solidification of a liquid therapeutic mixture. Non-limiting examples of such solidifying agents include a fatty acid base, hard fats, waxy solids, polyethylene glycol, poloxamers, or a combination thereof.

The therapeutic inserts or therapeutic mixture comprise one or more therapeutic compounds. The therapeutic inserts or therapeutic mixture can comprise any therapeutic compound that one of skill in the art would consider efficacious for promoting wound healing. In embodiments, the therapeutic compound comprises an antibiotic, an antihistamine, an anti-inflammatory agent, a coagulant, a steroid, an anti-fungal agent, an analgesic, a biofilm inhibitor, an angiogenic compound, a vasoconstrictive agent, a topical chemotherapeutic, tissue regenerative/enhancement agents, or a combination thereof. The therapeutic compound can also comprise broad spectrum antimicrobials such as chlorhexidine gluconate. In some embodiments, the antibiotic comprises clindamycin, vancomycin, or can be culture specific. The antifungal agent can comprise nystatin or any other appropriate antifungal agent. The anti-inflammatory agent can comprise betamethasone, hydrocortisone, corticosterone, prednisone, mometasone, or a combination thereof. The anti-inflammatory can also comprise a nonsteroidal anti-inflammatory (NSAID) drug such as aspirin, ibuprofen, celecoxib, ketorolac, or any other appropriate NSAID. The coagulant can comprise thrombin, prothrombin, avitene or any other appropriate coagulant. The vasoconstrictive agent can be amphetamines or antihistamines. Vasoconstrictive agents can include, but are not limited to: oxymetazoline, phenylephrine, epinephrine, xylometazoline, naphazoline, tetryzoline, angiotensin ii, vasopressin, felypressin, midodrine, methysergide, dihydroergotamine, norepinephrine, phenylpropanolamine, sumatriptan, ergotamine, pseudoephedrine, cocaine, lidocaine, tetracaine, ephedrine, levonordefrin, or any other appropriate vasoconstrictive agent. The topical chemotherapeutic can comprise fluorouracil, imiquimod, diclofenac, ingenol mebutate, or any other chemotherapeutic known by those of skill in the art to topically treat cancerous or pre-cancerous conditions. The tissue regenerative/enhancement agents can include growth hormones or growth factors. The therapeutic inserts, therapeutic mixture, therapeutic compounds, or a combination thereof can comprise a time-release mechanism to allow for controlled or sustained delivery of the therapeutic compound over a specific amount of time.

Also disclosed in another aspect is a method of administering one or more therapeutic compounds to a wound by use of a therapeutic device in accordance with any of the embodiments described in this disclosure. The method first provides inserting a therapeutic device into or near a wound on a subject in need thereof. Next, the method teaches allowing the therapeutic device to remain within or near the wound for a sufficient time to induce wound healing. In embodiments, the therapeutic device is inserted into the nasal cavity, the oral cavity, the ear, or any other bodily cavity. In embodiments, the therapeutic device is inserted simultaneously during or subsequently to a surgical procedure.

Also disclosed herein is a method for administering one or more pharmacological agents in preparation for a surgical procedure to a subject by use of a therapeutic device in accordance with any of the embodiments described in this disclosure.

An additional aspect includes a kit to promote wound healing. The kit employs a therapeutic device in accordance with any of the embodiments described in this disclosure. In embodiments, the kit includes the therapeutic device and instructions for use. The instructions can be physically included in the kit or provided on a remote server to which the subject or caretaker is given access.

In embodiments, the kit includes a therapeutic device that further comprises wound-packing material with one or more pockets, openings, or cavities as generally described herein. In these embodiments, the kit can further include a solidifying agent.

Also provided is a method for assembling a therapeutic device to promote wound healing. In this aspect, the method includes obtaining or providing a therapeutic device in accordance with any embodiment discussed herein, wherein the wound-packing material comprises one or more pockets, openings, or cavities. In embodiments, the method further includes obtaining or providing a solidifying agent. The method additionally provides mixing a therapeutic compound with the solidifying agent to create a therapeutic mixture. The method can also include filling the one or more pockets, openings, or cavities of the therapeutic device with the therapeutic mixture and allowing the therapeutic mixture to solidify into a suppository. In certain embodiments, the method further includes placing the therapeutic device within, on, or near a wound and allowing the suppository within the therapeutic device to melt or dissolve such that therapeutic compounds are released into or near the wound.

Other objects and advantages of this invention will become readily apparent from the ensuing description.

### BRIEF DESCRIPTION OF THE FIGURES

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1 shows a perspective cut-away view of the therapeutic device in accordance with one embodiment of the present invention. Three therapeutic inserts are shown extending longitudinally along a portion of the therapeutic device.

FIG. 2 shows a side view of a therapeutic insert in accordance with an embodiment of the present invention.

FIG. 3 shows a perspective view of a therapeutic device in accordance with an alternate embodiment of the present invention. Three therapeutic inserts are shown disposed within the therapeutic device as pellets or beads.

FIG. 4 shows a perspective view of the therapeutic device in accordance with yet another embodiment of the present invention. Three pockets are shown extending longitudinally along a portion of the therapeutic device.

### DETAILED DESCRIPTION OF THE INVENTION

### Abbreviations and Definitions

Detailed descriptions of one or more embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a,” “an,” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

For purposes of the present disclosure, it is noted that spatially relative terms, such as “up,” “down,” “right,” “left,” “beneath,” “below,” “lower,” “above,” “upper” and the like, can be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device can be otherwise oriented (rotated 90 degrees or at other orientations) and the spatially relative descriptors used herein interpreted accordingly.

The term “wound-packing material” includes without limitation any material or substance that absorbs drainage or exudate from a wound. The material can include, but is not limited to gauze, foam, packing strips, sponges, or any open-cell, absorbent, or porous material. The term includes any material that one having skill in the art would consider appropriate for packing wounds of various types, during various healing phases, and in various locations across or within the body.

The terms “non-adherent coating” or “non-adherent dressing” includes without limitation any material or substance that prevents wound-packing material from sticking to the wound or surrounding tissue. This term, for example, encompasses low-adherent dressings and reduced-adherent dressings, or the substantial equivalents thereof.

The terms “subject” and “patient” as used herein include all members of the animal kingdom including, but not limited to, mammals, animals (e.g., cats, dogs, horses, swine, etc.) and humans.

The term “caretaker” as used herein refers to any person, group, or entity who has assumed responsibility to care for the subject or patient or to prepare the therapeutic device for use. By way of non-limiting example, a caretaker can include a physician, a nurse, a clinician, a pharmacist, a physician's assistant, any employee of a clinical facility, a family member of the subject, a friend or acquaintance of the subject, an employee of the subject, or any other person, group, or entity who assumes responsibility to care for the subject. In certain instances, the subject can act as a caretaker, such as when tending to his or her own wounds.

The terms “biodegradable,” “bioabsorbable,” and “bioerodable” are used interchangeably throughout this disclosure and refer to materials capable of breaking down within the subject through natural biological processes. Upon dissolution, such materials are processed into small, nontoxic compounds that are capable of removal via natural clearance mechanisms. These materials can be broken down through chemical reactions or can be dissolved in a manner devoid of chemical alterations of the molecular structure.

The term “therapeutic mixture” can refer to any material or substance or combination of materials or substances that includes at least one therapeutic compound. Therapeutic mixtures can exist in solid or liquid form and can be solid at room temperature and liquid when heated. The therapeutic mixture can include a solidifying agent that is configured to promote solidification of the therapeutic mixture in a time-dependent or temperature-dependent manner. A therapeutic mixture may exist as a suppository-type substance that is configured to melt or dissolve at a certain temperature or after a specified amount of time.

### Description of Selected Embodiments

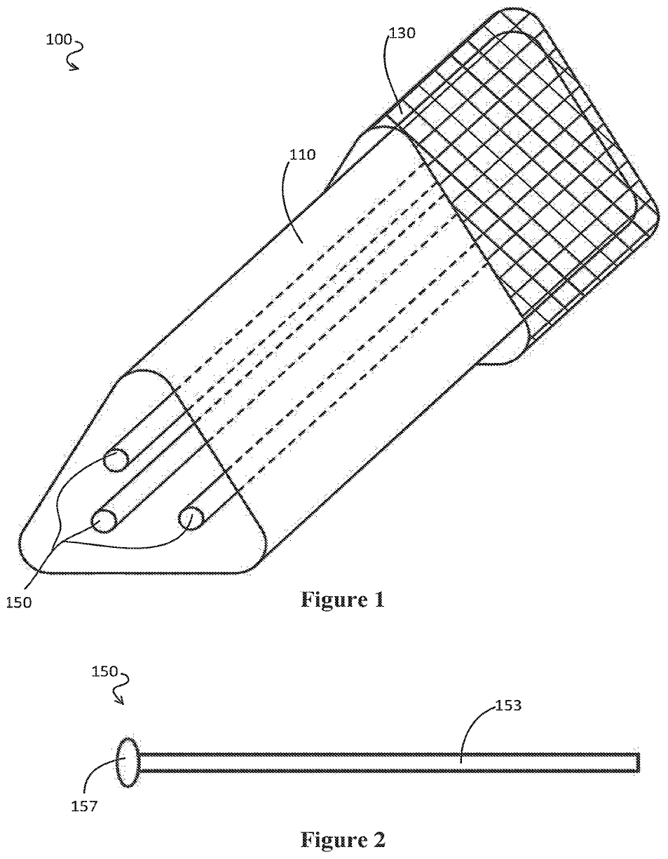

FIG. 1 shows an embodiment of the therapeutic device 100 with a portion of one end cut away. The embodiment comprises a triangular-shaped cylindrical body 110. The body of the device is made up of wound-packing material 110. As shown, the corners of the therapeutic device are rounded. Alternate embodiments can comprise straight edges or corners. Although a triangular-shaped, cylindrical body 110 is provided in the FIG. 1 embodiment, a variety of cross-sectional shapes are envisioned in alternate embodiments of the present invention. The cross-sectional shape of the therapeutic device 100 will vary depending on needs or preferences of the subject or caretaker. The size, shape, or configuration of the therapeutic device 100 also varies with the wound type, size, and location. In certain embodiments, the size and shape of the therapeutic device are fully customizable (discussed in more detail below), allowing the device to be molded, shaped, or trimmed to the desired specifications.

The cut away view of FIG. 1 reveals three therapeutic inserts 150 residing within the wound-packing material 110. The therapeutic inserts 150 of the FIG. 1 embodiment are generally rod-like and extend longitudinally across at least a portion of the length of the wound-packing material 110. In embodiments, the therapeutic inserts 150 extend between about ¼ to about ¾ of the longitudinal length of the therapeutic device 100. The inserts 150 can extend between about ⅓ to about ⅔ of the longitudinal length of the therapeutic device 100. Alternatively, the inserts 150 can extend about halfway across the length of the therapeutic device 100. In still other embodiments, the therapeutic inserts 150 can extend across the entire length or a substantial portion of the therapeutic device 100.

In the FIG. 1 embodiment, the therapeutic device 100 comprises perforations that are configured to frictionally secure the therapeutic inserts 150 within the wound-packing material 110. In embodiments, the perforations can include gaps, holes, slots, notches, grooves, or a combination thereof. A number of alternate or additional mechanisms for securing the therapeutic inserts 150 within the wound-packing material would be apparent to one of skill in the art. By way of non-limiting example, the therapeutic inserts 150 can be secured to the wound-packing material via glue, tape, sutures, staples, clamps, or a combination thereof. In certain embodiments, a bio-adhesive or medical-grade tape secures the therapeutic inserts to the wound-packing material 110.

Although three therapeutic inserts 150 are pictured in the FIG. 1 embodiment, the number of therapeutic inserts can vary. Embodiments can comprise between one and ten therapeutic inserts 150, inclusive. Alternate embodiments can comprise between one and five therapeutic inserts 150, inclusive. In embodiments, there can be one, two, three, four, five, six, seven, eight, nine, or ten therapeutic inserts 150. In certain embodiments, the number of therapeutic inserts 150 is customizable depending the needs or preferences of the subject or caretaker.

A non-adherent coating 130 is shown surrounding a portion of the PVA sponge 110 in FIG. 1. The non-adherent coating 130 prevents the therapeutic device from sticking to the wound or to the surrounding tissue during the healing process. This allows for easier removal of the device and increases patient comfort when wound dressings are changed. In embodiments, the non-adherent coating 130 completely surrounds the wound-packing material 110. In certain embodiments, the non-adherent coating 130 surrounds about ¼ to about ¾ of the wound-packing material 110. The non-adherent coating 130 can surround about ⅓ to about ⅔ of the wound-packing material 110. Alternatively, the inserts 130 can cover about half of the wound-packing material 110.

FIG. 2 shows a side view of a therapeutic insert 150 in accordance with one embodiment of the present invention. A flattened disc 157 is shown at one end of the insert and a single rod 153 is shown extending longitudinally away from the disc 157. The flattened dis 157 is configured to firmly secure the therapeutic insert 150 within the therapeutic device 100. Although the therapeutic insert 150 is shown comprising a cylindrical body with a generally circular cross section, alternate cross-sectional shapes are also envisioned. For instance, the cross-section of the cylindrical body can be substantially oval or polygonal in shape. In polygonal embodiments, the cross section can comprise shapes that are regular or irregular polygons. Embodiments of the therapeutic insert 150 are substantially triangular in shape, substantially rectangular in shape, or substantially square. Embodiments can comprise straight or curved edges. In still other embodiments, the therapeutic insert 150 can comprise a spherical, conical, or disc-like body. The body of the therapeutic insert 150 can be structurally rigid or can be pliable to allow for customization of the design of the therapeutic device 100.

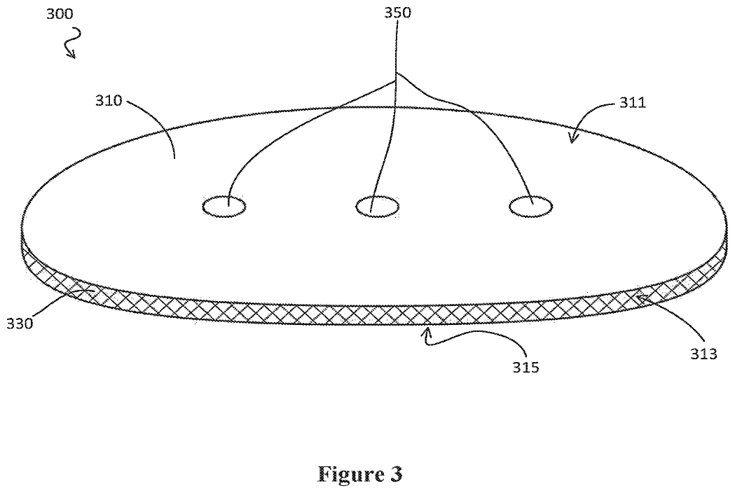

FIG. 3 provides a therapeutic device 300 in an alternate aspect. In the FIG. 3 aspect, the therapeutic device 300 comprises a cylindrical body 310 that is generally flattened or disc-shaped, and the disc-shaped body is comprised of wound-packing material 310. Although the FIG. 3 embodiment comprises a circular, disc-shaped body 310, a variety of cross-sectional shapes are suitable in alternate, flattened embodiments of the present invention. The cross-sectional shape of the therapeutic device 300 varies depending on needs or preferences of the subject or caretaker. The size, shape, or configuration of the therapeutic device 300 also varies with the wound type, size, or location. Disc-shaped embodiments, such as the FIG. 3 aspect, are particularly useful for treatment of large surface wounds or lesions. Such embodiments of the therapeutic device 300 may be configured to reside within, reside upon, or cover a surface wound or lesion. Such lesions can include cancerous or precancerous lesions. Non-limiting examples of pre-cancerous lesions include actinic keratosis, actinic cheilitis. Examples of cancerous lesions include but are not limited to melanoma, basal cell carcinoma, or squamous cell carcinoma.

The FIG. 3 aspect reveals three therapeutic inserts 350 residing within the wound-packing material 310. The therapeutic inserts 350 of the FIG. 3 embodiment comprise beads or pellets, which are generally spherical in shape. However, the therapeutic inserts 350 may comprise any shape or configuration as discussed herein. The inserts 350 may be secured within the therapeutic device 300 as generally described with regard to the FIG. 1 embodiment. Additionally, the number of therapeutic inserts 350 pictured in FIG. 3 may vary and may be customizable, as discussed herein with regard to the FIG. 1 embodiment.

A non-adherent coating or surface 330 is shown surrounding a portion of the wound-packing material 310 in FIG. 3. In the FIG. 3 embodiment, the non-adherent coating 330 covers an undersurface 315 of the therapeutic device 300 and is shown continuing along the sides 313 of the therapeutic device 300. In certain embodiments, the non-adherent coating 330 covers only the undersurface 315 of the therapeutic device 300. The non-adherent coating can cover the top 311, sides 315, undersurface 315, or a combination thereof of the therapeutic device 300. In embodiments, the non-adherent coating completely surrounds the wound-packing material 310 to cover the top 311, sides 315, and undersurface 315 of the therapeutic device 300.

The therapeutic inserts 150, 350 are comprised of materials that permit controlled release of therapeutic compounds to target tissues over a specified time period. In embodiments, the inserts 150, 350 are comprised of a biodegradable, bioerodible, or bioabsorbable polymer material. Examples of such materials include, but are not limited to polyethylene glycol, poly trimethylene carbonate, polycaprolactone, self-reinforced polyglycolide, or self-reinforced polylactide. In certain embodiments, the therapeutic inserts are comprised of glyceride, synthetic glyceride, semi-synthetic glyceride, or a combination thereof.

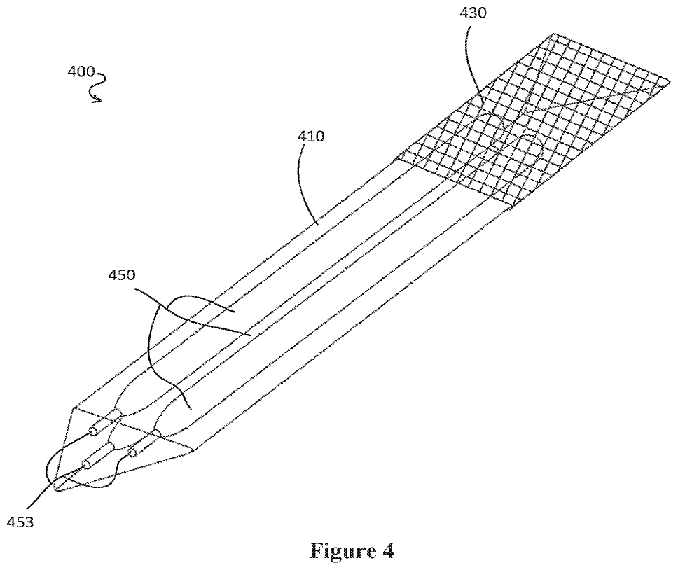

FIG. 4 shows another aspect of the therapeutic device 400 in an alternate configuration. Although a triangular-shaped, cylindrical body 410 is provided in the FIG. 4 embodiment, a variety of cross-sectional shapes are envisioned in alternate embodiments. The cross-sectional shape of the therapeutic device 400 will vary depending on needs or preferences of the subject or caretaker. The size, shape, or configuration of the therapeutic device 400 also varies with the wound type, size, and location. In certain embodiments, the size and shape of the therapeutic device 400 are fully customizable (discussed in more detail below), allowing the device 400 to be molded, shaped, or trimmed to the desired specifications.

A non-adherent coating 430 is shown surrounding a portion of the wound-packing material 410 in FIG. 4. In embodiments, the non-adherent coating 430 completely surrounds the wound-packing material 410. In certain embodiments, the non-adherent coating 430 surrounds about ¼ to about ¾ of the wound-packing material 410. The non-adherent coating 430 can surround about ⅓ to about ⅔ of the wound-packing material 410. Alternatively, the inserts 430 can cover about half of the wound-packing material 410.

Also shown in FIG. 4, the therapeutic device 400 comprises wound-packing material 410 that further comprises one or more pockets, openings, or cavities 450. The pockets, openings, or cavities are configured to receive a therapeutic mixture that comprises one or more therapeutic compounds. In embodiments, the pockets, openings, or cavities 450 include a tapering that terminates in a narrowed portion 453 at one end of the pocket, opening, or cavity. The narrowed portion 453 serves as a port, a channel, or a point of injection to allow for introduction of the therapeutic mixture into the pocket, opening, or cavity of the therapeutic device. Although the pockets, openings, or cavities 450 are shown comprising a cylindrical body with a generally circular cross section, alternate cross-sectional shapes are also envisioned, as detailed above with regard to the therapeutic inserts 150.

In the FIG. 4 aspect, the pockets, openings, or cavities 450 extend longitudinally or horizontally or across at least a portion of the length or height of the wound-packing material 410. In embodiments, the pockets, openings, or cavities 450 extend between about ¼ to about ¾ of the longitudinal length or height of the therapeutic device 400. The pockets, openings, or cavities 450 can extend between about ⅓ to about ⅔ of the longitudinal length or height of the therapeutic device 400. Alternatively, the pockets, openings, or cavities 450 can extend about halfway across the length or height of the therapeutic device 400. In still other embodiments, the pockets, openings, or cavities 450 can extend across the entire length, height, or a substantial portion of the therapeutic device 400. Embodiments can comprise between one and ten pockets, openings, or cavities 450, inclusive. Alternate embodiments can comprise between one and five pockets, openings, or cavities 450, inclusive. In embodiments, there can be one, two, three, four, five, six, seven, eight, nine, or ten pockets, openings, or cavities 450. In certain embodiments, the number of pockets, openings, or cavities 450 is customizable depending the needs or preferences of the subject or caretaker.

In certain embodiments of the FIG. 4 aspect, the therapeutic mixture is substantially liquid before introduction into the therapeutic device 400. The liquid therapeutic mixture can also include a solidifying agent that is configured to promote solidification of the therapeutic mixture after the mixture is introduced into the pockets, openings, or cavities 450 of the therapeutic device 400. In embodiments, the solidified therapeutic mixture forms a suppository-type substance within the therapeutic device 400 that is configured to melt or dissolve in response to contact with a wound for controlled release of therapeutic compounds into the wound. The therapeutic mixture can be configured to melt at a wide range of temperatures. In embodiments, the therapeutic mixture is configured to melt at temperatures between about 78° F. and about 120° F. The therapeutic mixture can be configured to melt at temperatures between about 85° F. and about 110° F. In certain embodiments, the therapeutic mixture melts at about 86° F., about 87° F., about 88° F., about 89° F., about 90° F., about 91° F., about 92° F., about 93° F., about 94° F., about 95° F., about 96° F., about 97° F., about 98° F., about about 99° F., about 100° F., about 101° F., or about 102° F.

The solidifying agent can be comprised of any agent, compound, or material known by those of skill in the art or later developed that promotes solidification of a liquid therapeutic mixture. Non-limiting examples of such solidifying agents include a fatty acid base, hard fats, waxy solids, polyethylene glycol, poloxamers, or a combination thereof. The hard fats can include any fatty substance that is solid a room temperature. In embodiments, the hard fats are configured to melt when placed within, on or near a wound or surgical incision. The hard fats can melt at or below the body temperature of the subject. The hard fats can be configured to melt when placed within a bodily cavity. Examples of hard fats include but are not limited to cocoa butter, synthetic triglyceride mixtures, or any other hard fat known by those in the art to be appropriate for forming suppositories. Hard fats can be comprised of any triglyceride, diglyceride, monoglyceride, or a combination thereof. Non-limiting examples of waxy solids include cetyl alcohol, stearic acid, stearyl alcohol, cetostearyl alcohol, myristyl alcohol, or a combination thereof. The poloxamers can be poloxamer 188, poloxamer 407, or any other poloxamer known by a skill artisan to provide a suitable stiffening agent for a therapeutic mixture.

The polymer material or therapeutic mixture can be configured to undergo surface degradation, bulk degradation, or a combination of both. When undergoing surface degradation, the exterior surface of the polymer is progressively broken down until the polymer is completely degraded, resulting in a reduction of the physical size of the insert as the outer lay of the polymer dissolves. In bulk degradation, both the exterior surface and the interior of the polymer material erode simultaneously. Thus, when undergoing bulk degradation, the volume of the therapeutic insert remains fairly consistent until the polymer is almost fully degraded. Surface degradation, bulk degradation, or a combination thereof can provide a mechanism to allow for controlled or sustained delivery of the therapeutic compound over a specific amount of time.

The therapeutic inserts 150, 350 or therapeutic mixture comprise therapeutic compounds. In embodiments, the therapeutic inserts are coated, infused, or impregnated with the therapeutic compounds. In embodiments, the therapeutic inserts 150, 350 or therapeutic mixture comprises more than one type of therapeutic compound. The therapeutic insert 150, 350 or therapeutic mixture can comprise between one and ten different therapeutic compounds. In embodiments, the therapeutic insert 150, 350 or therapeutic mixture can comprise between two and five different therapeutic compounds. The insert 150, 350 or mixture can comprise two, three, four, five, six, seven, eight, nine, or ten different therapeutic compounds. In one embodiment, the therapeutic insert 150, 350 or therapeutic mixture comprises a single type of therapeutic compound.

The therapeutic compounds can comprise hydrophobic agents, hydrophilic agents, or a combination of both. In one embodiment, the therapeutic insert 150, 350 or therapeutic mixture comprises therapeutic compounds that promote wound healing. Examples of such compounds include, but are not limited to an antibiotic, an antihistamine, an anti-inflammatory agent, a coagulant, a steroid, an anti-fungal agent, an angiogenic compound, a vasoconstrictive agent, an analgesic, a biofilm inhibitor, a topical chemotherapeutic, tissue regenerative agents, or a combination thereof. The therapeutic compound can further comprise bioactive molecules such as growth hormones or growth factors. Such growth factors include, but are not limited to epidermal growth factor, transforming growth factor, vascular endothelial growth factor, fibroblast growth factor, platelet-derived growth factor, interleukins, colony-stimulating factors, keratinocyte growth factor, or a combination thereof. The therapeutic compound can also comprise broad spectrum antimicrobials such as chlorhexidine gluconate. Examples of potential antibiotics include, but are not limited to clindamycin or vancomycin. In embodiments, the antibiotic is culture-specific. The antifungal agent can be any appropriate antifungal agent including, but not limited to, nystatin. Non-limiting examples of the anti-inflammatory agent include betamethasone, hydrocortisone, corticosterone, prednisone, a combination thereof. The anti-inflammatory can also comprise a nonsteroidal anti-inflammatory (NSAID) drug such as aspirin, ibuprofen, celecoxib, ketorolac, or any other appropriate NSAID. In embodiments with a coagulant, the coagulant can comprise thrombin, prothrombin, avitene, or any other appropriate coagulant. The vasoconstrictive agent can comprise amphetamines or antihistamines. The topical chemotherapeutic can include fluorouracil, imiquimod, diclofenac, ingenol mebutate, or any other topical chemotherapeutic or combination of topical chemotherapeutic known to those of skill in the art.

In embodiments, vasoconstrictors mixed with local anesthetics can be used to increase the duration of local anesthesia by constricting the blood vessels. Thus, the anesthetic agent can be concentrated for an extended duration, and can, for example, reduce hemorrhage events. Vasoconstrictor agents can be amphetamines or antihistamines. Non-limiting examples of vasoconstrictive agents include: oxymetazoline, phenylephrine, epinephrine, xylometazoline, naphazoline, tetryzoline, angiotensin ii, vasopressin, felypressin, midodrine, methysergide, dihydroergotamine, norepinephrine, phenylpropanolamine, sumatriptan, ergotamine, pseudoephedrine, cocaine, lidocaine, tetracaine, ephedrine, levonordefrin, or any other appropriate vasoconstrictive agent.

The wound-packing material 110, 310, 410 can comprise various substances that are appropriate for absorbing drainage from a wound. The wound-packing material can comprise hydrophilic materials. In one embodiment, the wound-packing material comprises a polyvinyl alcohol (PVA) sponge. In an alternate embodiment, the wound-packing material comprises a collagen dressing to stimulate tissue growth during the proliferation stage of wound healing.

Another aspect of the present invention includes a method of administering one or more therapeutic compounds to a wound by use of a therapeutic device 100, 300, 400 in accordance with any embodiment disclosed within this specification or otherwise apparent from the descriptions herein. In embodiments, the therapeutic device 100, 300, 400 is inserted into or near a wound of a subject. In certain embodiments, the therapeutic device 100, 300, 400 is placed over or covers a surface wound, blemish, or lesion. The therapeutic device 100, 300, 400 is then allowed to remain within or near the wound for sufficient time to induce wound healing. In embodiments, the therapeutic device 100, 300, 400 is inserted simultaneously during or subsequently to a surgical procedure. In one embodiment, the therapeutic device 100, 300, 400 is inserted into the nasal cavity, the oral cavity, the ear, or any other bodily cavity.

In one embodiment, the therapeutic device 100, 300, 400 remains within, on, over, or near the wound until the therapeutic compounds in the therapeutic insert 150, 350 or therapeutic mixture has sufficiently diffused into the wound. In certain embodiments, the therapeutic device 100, 300 remains within, on, over, or near the wound until the biodegradable therapeutic inserts 150, 350 or therapeutic mixture is completely degraded. In other embodiments, the therapeutic device 100, 300 remains in, on, or near the wound until the therapeutic inserts 150, 350 or therapeutic mixture is volumetrically reduced by between 10% to about 90%, inclusive. The therapeutic device 100, 300 can remain in, on, or near the wound until a volumetric reduction of the therapeutic inserts 150, 350 or therapeutic mixture from about 30% to about 50% is achieved. In embodiments, the volumetric reduction can be about 10%, 20%, 30%, 40%, 50%, 60%, 70%, 80% or 90% before the therapeutic device 150, 350 is removed.

In alternate embodiments, the therapeutic device 100, 300, 400 remains within, on, or near the wound for a specific amount of time. The device 100, 300, 400 can remain in or near the wound for a period of up to one month. In embodiments, the device 100, 300, 400 can remain in or near the wound for less than one week. The therapeutic device 100, 300, 400 can remain in or near the wound for one, two, three, four, five, six, seven, or eight days.

Also disclosed is a kit that includes the therapeutic device 100, 300, 400 in accordance with any embodiment disclosed within this specification or otherwise apparent from the descriptions herein. In embodiments, the kit comprises the therapeutic device 100, 300, 400 and instructions for use or assembly of the therapeutic device 100, 300. The instructions can be physically provided with the kit or accessible separately from the kit, such as via the retailer's or manufacturer's website. In embodiments, the kit includes an unassembled or partially assembled therapeutic device 100, 300, 400. The non-adherent coating 130, 330, 430 can be provided already surrounding the wound-packing material 110, 310, 410 or added later. In embodiments, the kit does not include the non-adherent coating 110, 310, 410, and the kit recipient selects a non-adherent coating 130, 330 of their choice.

Aspects that comprise a kit are particularly advantageous for customizable therapeutic devices 100, 300, 400. In certain customizable embodiments, the wound-packing material110, 310, 410, is provided as a blank of polymer material that is capable of being trimmed, molded, or otherwise shaped for the desired application of the therapeutic device 100, 300, 400. For instance, a customizable intranasal therapeutic device 100, 300, 400 can be provided, wherein the dimensions of the therapeutic device 100, 300, 400 are adjustable to fit the individual variances in nasal cavity size. Such a customizable therapeutic device 100, 300, 400 is not limited to use within the nasal cavity. Other embodiments are customizable to fit any bodily cavity, including, but not limited to the oral cavity, the ear canal, middle ear, or inner ear. The therapeutic device 100, 300, 400 can also be customizable based on the size, shape, or type of the wound or surgical incision. The therapeutic device 100, 300, 400 can be customizable based on the subject's specific needs, preference, and comfort.

In customizable embodiments, the blank of material can comprise a variety of sizes and shapes from which the physician, nurse, clinician, or other caretaker can select as a starting point for further customization of the therapeutic device 100, 300, 400. The blank of polymer material can be substantially polyhedral or cylindrical.

Embodiments of the kit comprise either pre-assembled or ready-to-assemble therapeutic inserts 150, 350. In certain embodiments, the kit includes the necessary ingredients/materials for construction of the therapeutic insert 150, 350. The necessary ingredients/materials can include any of the following: polymer material, molds for casting the shape of the therapeutic insert, therapeutic compounds, a frame to which the polymer material or therapeutic compound adheres, or tools to aid construction of the insert. The necessary ingredients/materials can be pre-measured/pre-weighed or provided as a bulk from which the kit recipient determines the appropriate amount.

In certain embodiments, the kit includes therapeutic inserts 150, 350 or a therapeutic mixture with no therapeutic compounds incorporated within or on the inserts 150, 350 or mixture. Such embodiments permit the kit recipient to tailor the therapeutic device 100, 300, 400 to the needs or preferences of the subject or caretaker. In embodiments, the therapeutic compounds can be incorporated onto or within the therapeutic insert 150, 350 via compression, spray, dip coating, encapsulation, or any other method known in the art. The therapeutic inserts or therapeutic mixture can be included within the kit or can be provided separately therefrom.

In embodiments, the kit includes a therapeutic device 400 in accordance with the FIG. 4 aspect, wherein the wound-packing material 410 comprises one or more pockets, openings, or cavities 450. In these embodiments, the kit can further include a solidifying agent. The solidifying agent can be provided as a base of material that is solid at room temperature. In embodiments the kit provides instructions for converting the solidifying agent into a therapeutic mixture that comprises one or more therapeutic compounds. Certain embodiments of the kit further include at least one therapeutic compound, while other embodiments are provided without a therapeutic compound. Kit embodiments without a therapeutic compound allow the caretaker or subject to select an appropriate therapeutic compound for use within the therapeutic device 400. Embodiments of the kit may further comprise a stirring device or stirrer to improve the distribution of the therapeutic compound within the therapeutic mixture.

In the various exemplary embodiments, the size of therapeutic device can be configured to fit with any of various bodily cavities. In one embodiment, the therapeutic device is configured to fit within the nasal cavity, the oral cavity, the ear, or any other bodily cavity. The therapeutic device can be configured to within any of various wounds or sizes of wounds. In certain embodiments, the therapeutic device is configured to cover a wound. The length of the therapeutic device can be between about 1 mm to about 1000 mm long, inclusive. The therapeutic device can be up to about 500 mm long. The therapeutic device can be about 300 mm or less. The therapeutic device can be less than 200 mm long. The therapeutic device can be less than 100 mm in length. In embodiments, the length of the therapeutic device is between about 5 mm to about 25 mm, inclusive. In certain embodiments the therapeutic device is less than 5 mm long.

Also provided is a method for assembling a therapeutic device to promote wound healing. In this aspect, the method includes obtaining a therapeutic device 100, 300, 400 in accordance with any embodiment discussed herein. In certain embodiments, the therapeutic device is in accordance with FIG. 4, wherein the wound-packing material 410 comprises one or more pockets, openings, or cavities 450. In embodiments, the method further includes obtaining a solidifying agent. The method additionally provides mixing a therapeutic compound with the solidifying agent to create a therapeutic mixture. In certain embodiments, the method includes melting the solidifying agent before adding the therapeutic compound to create the therapeutic mixture. The solidifying agent may be melted via any appropriate methodology. By way of non-limiting example, the solidifying agent may be melted in a microwave, over a hot plate, over a burner, over an open flame, or any combination thereof. The method can also include filling the one or more pockets, openings, or cavities 450 of the therapeutic device 400 with the liquid therapeutic mixture and allowing the therapeutic mixture to solidify into a suppository. The liquid therapeutic mixture can be injected or otherwise inserted into the one or more pockets, openings, or cavities 450 of the therapeutic device 400. In certain embodiments, the method further includes placing the therapeutic device 400 within, on, or near a wound and allowing the suppository within the therapeutic device 40 to melt or dissolve such that therapeutic compounds are released into or near the wound.

An additional aspect of the present invention includes a device for delivery of pharmaceutical agents in preparation for a surgical procedure. Pre-surgical aspects comprise a therapeutic device 100, 300400, in accordance with any embodiment disclosed within this specification or otherwise apparent from the descriptions herein. In embodiments of the pre-surgical aspect, the inserts 150, 350 or mixture comprises any agents known by those of skill in the art to be beneficial in preparing a patient for surgery. Examples of such agents include, but are not limited to analgesics and anesthetics. In embodiments, pre-surgical devices 100, 300 will be placed within a bodily cavity and adjacent to the designated surgical site.

Another aspect of the present invention includes a method for administering one or more pharmacological agents in preparation for a surgical procedure to a subject by use of a therapeutic device 100, 300, 400 in accordance with any of the embodiments described in this disclosure.

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

1. A therapeutic device to promote wound healing, wherein the therapeutic device comprises:
wound-packing material comprising one or more gaps, slots, holes, notches, grooves, or a combination thereof that are configured to hold one or more therapeutic inserts;
a non-adherent, absorbent coating surrounding at least a portion of the wound-packing material; and
a plurality of therapeutic inserts configured to reside within the wound-packing material.

2. The device of claim 1, wherein the therapeutic device is configured to reside within a bodily cavity or over a surf ace wound or lesion.

3. The device of claim 2, wherein the therapeutic device is configured to reside within a nasal cavity.

4. (canceled)

5. (canceled)

6. (canceled)

7. (canceled)

8. The device of claim 1, wherein the wound-packing material comprises a polyvinyl alcohol (PVA) sponge,

9. (canceled)

10. (canceled)

11. (canceled)

12. The device of claim 1, wherein the therapeutic insert is configured to reside across a longitudinal length of the wound-packing material.

13. The device of claim 1, wherein the therapeutic insert is a therapeutic rod, a therapeutic bead, or a combination thereof.

14. (canceled)

15. The device of claim 1, wherein the therapeutic insert comprises a bio-absorbable polymer material.

16. The device of claim 1, wherein the plurality of therapeutic inserts comprise one or more therapeutic compounds.

17. The device of claim 16, wherein the therapeutic compounds comprises an antibiotic, an antihistamine, an anti-inflammatory agent, a coagulant, a steroid, an anti-fungal agent, an analgesic, a biofilm inhibitor, an angiogenic compound, a vasoconstrictive agent, a topical chemotherapeutic, tissue regenerative agents, or a combination thereof.

18. (canceled)

19. (canceled)

20. (canceled)

21. An intranasal therapeutic device to promote wound healing, wherein the therapeutic device comprises:
a triangular-shaped, polyvinyl alcohol (PVA) sponge, wherein the PVA sponge comprises one or more gaps, slots, holes, notches, grooves, or a combination thereof that continue longitudinally across a portion of the PVA sponge, and wherein the gaps, slots, holes, notches, grooves, or a combination thereof are configured to receive one or more therapeutic inserts;
a non-adherent, absorbent material surrounding at least a portion of the PVA sponge;
a plurality of therapeutic inserts configured to reside within the PVA sponge, wherein the plurality of therapeutic inserts are comprised of a bio-absorbable polymer material; and wherein the plurality of therapeutic inserts further comprise one or more therapeutic compounds, wherein the one or more therapeutic compounds comprise an antibiotic, an antihistamine, an anti-inflammatory agent, a coagulant, a steroid, an anti-fungal agent, an analgesic, a biofilm inhibitor, an angiogenic compound, a vasoconstrictive agent, a topical chemotherapeutic, a tissue regenerative agent, or a combination thereof.

22. The device of claim 17, wherein the antibiotic comprises clindamycin or vancomycin and the antifungal agent comprises nystatin.

23. The device of claim 17, wherein the anti-inflammatory agent comprises betamethasone, hydrocortisone, corticosterone, prednisone, or a combination thereof.

24. The device of claim 17, wherein the anti-inflammatory agent comprises a nonsteroidal anti-inflammatory agent.

25. The device of claim 17, wherein the coagulant comprises thrombin, prothrombin, avitene, or a combination thereof.

26. The device of claim 17, wherein the biofilm inhibitor is chlorhexidine gluconate.

27. The device of claim 17, wherein the topical chemotherapeutic comprises fluorouracil, imiquimod, diclofenac, ingenol mebutate, or a combination thereof.

28. The device of claim 17, wherein the tissue regenerative agent comprises a growth hormone, a growth factor, or a combination thereof.

29. The device of claim 17, wherein the bio-absorbable polymer material of the plurality of therapeutic rods comprises polyethylene glycol, poly trimethylene carbonate, polycaprolactone, self-reinforced polyglycolide, or self-reinforced polylactide.

30. The device of claim 17, wherein the vasoconstrictive agent comprises oxymetazoline, phenylephrine, epinephrine, xylometazoline, naphazoline, tetryzoline, angiotensin ii, vasopressin, felypressin, midodrine, methysergide, dihydroergotamine, norepinephrine, phenylpropanolamine, sumatriptan, ergotamine, pseudoephedrine, cocaine, lidocaine, tetracaine, ephedrine, levonordefrin, or a combination thereof.

31. The device of claim 1, wherein the therapeutic device further comprises a customizable shape, size, or combination thereof.

32. A method of administering one or more therapeutic compounds to a wound in a subject, the method comprising inserting the therapeutic device of claim 1 into, on, or near a wound on a subject in need thereof and allowing the therapeutic device to remain therein or thereon for a sufficient time to induce wound healing.

33. The method of claim 32, wherein the therapeutic device is inserted into a bodily cavity or placed over a surface wound or lesion.

34. (canceled)

35. The method of claim 32, wherein the therapeutic device is inserted simultaneously during or subsequently to a surgical procedure.

36. (canceled)

37. (canceled)

38. A method of intranasal administration of therapeutic compounds, the method comprising inserting the therapeutic device of claim 1 into a nasal cavity of a subject following a surgical procedure and allowing the therapeutic device to remain therein for a sufficient time to induce wound healing or until the therapeutic inserts are absorbed.

39. (canceled)

40. (canceled)

41. (canceled)

42. A therapeutic device to promote wound healing, wherein the therapeutic device comprises:
wound-packing material, the wound-packing material further comprises one or more pockets, openings, or cavities configured to receive a therapeutic mixture or therapeutic insert;
a non-adherent, absorbent coating surrounding at least a portion of the wound-packing material.

43. (canceled)

44. The device of claim 42, wherein the therapeutic mixture comprises one or more therapeutic compounds and a solidifying agent configured to form a suppository.

45. The device of claim 44, wherein the suppository is configured to melt or dissolve when applied within, on, or over a wound.

46. The device of claim 44, wherein the solidifying agent comprises a fatty acid base, a hard fat, a waxy solid, polyethylene glycol, a polyaxmer, or a combination thereof.

47. (canceled)

48. (canceled)

49. A method for assembling a therapeutic device comprising:
obtaining the therapeutic device off claim 42;
obtaining a solidifying agent;
mixing a therapeutic compound with the solidifying agent to create a therapeutic mixture;
filling the one or more pockets, openings, or cavities with the therapeutic mixture; and
allowing the therapeutic mixture to solidify into a suppository.

50. (canceled)

51. (canceled)
